(19) United States
(12) Patent Application Publication
(10) Pub. No.: US 2007/0020212 A1
(43) Pub. Date: Jan. 25, 2007

(54) System and method to promote proper handwashing

(76) Inventors: Jesse Bernal, Carlsbad, CA (US); Shahnawaz Khatri, Carlsbad, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/185,235

(22) Filed: Jul. 19, 2005

(51) Int. Cl. A61K 8/00

(52) U.S. Cl. 424/70.1

(57) ABSTRACT

The present invention generally relates to systems and methods to promote proper handwashing. Specifically, it relates to systems and methods that provide feedback to the person who is washing his hands. It further relates to systems and methods for monitoring hand hygiene compliance. In a system aspect of the present invention, a system for promoting adherence to handwashing guidelines is provided. The system includes: a sensor for detecting a handwashing event; a controller that receives signals from the sensor and activates a timer; a timer for sending an observable signal to a handwasher; and, a power source that provides power to components of the system.

### FIELD OF THE INVENTION

The present invention generally relates to systems and methods to promote proper handwashing. Specifically, it relates to systems and methods that provide feedback to the person who is washing his hands. It further relates to systems and methods for monitoring hand hygiene compliance.

### BACKGROUND OF THE INVENTION

The spread of biological pathogens can be inhibited or prevented in a number of ways. The simplest way, however, involves adequate hand washing. That is due to the fact that transmission of bacteria readily occurs from the hands of someone who has touched a contaminated material to another treated by those hands, especially in the healthcare field. Bacterial contamination, furthermore, is reduced or eliminated by following straightforward guidelines related to hand hygiene. See, for example, “Dr. Semmelweiss Was Right: Washing Hands Prevents Infection.” Water Quality and Health website. Available at: www.waterandhealth.org/newsletter/new/feb.-1998.

Convincing, or even attempting to compel, workers to follow simple guidelines, is difficult though. Reports indicate that healthcare workers adhere to hand washing guidelines less than 70% of the time. See, O'Boyle, C. A., Henly, S. J., Larson, E. “Understanding adherence to hand hygiene recommendations: the theory of planned behavior.” Am J Infect Control. 2001, 29(6):352-360. Certain groups of professionals, such as dentists, may do slightly better. See, McCarthy G. M., Koval J. J., John M. A., McDonald J. K. “Infection control practices across Canada: do dentists follow the recommendations?” J Can Dent Assoc 1999; 65:506-11.

Studies suggest a variety of reasons why hand hygiene guidelines are not followed. The reason most oftentimes cited is that workers either do not have or do not want to take the time to properly wash their hands. See, Widmer A. F. “Infection control and prevention strategies in the ICU.” Intensive Care Med 1994; 20(Suppl 4):S7-11. This may not be the primary cause, though, given that many healthcare workers believe they are complying with guidelines. See Peters, D. A., et al. “Hands that heal, or hands that harrn?” Career Management. Available at www.community.nursingspectrum.com.

Whatever the cause of non-adherence, the Center for Disease Control views hand hygiene so seriously that it has issued a fact sheet and a set of recommendations for healthcare facilities. See, CDC website. Available at www.cdc.gov. As part of the recommendations, the CDC is asking healthcare facilities to develop and implement a system for measuring improvements in following hand hygiene guidelines. Some of the suggested performance indicators include: periodic monitoring of hand hygiene adherence and providing feedback to personnel regarding their performance; monitoring the volume of alcohol-based handrub used per 1000 patient days; and, focused assessement of the adequacy of healthcare personnel hand hygiene when outbreaks of infection occur.

In view of the above, new systems and methods for promoting proper handwashing are needed. That is an object of the present invention.

### SUMMARY OF THE INVENTION

The present invention generally relates to systems and methods to promote proper handwashing. Specifically, it relates to systems and methods that provide feedback to the person who is washing his hands. It further relates to systems and methods for monitoring hand hygiene compliance.

In a system aspect of the present invention, a system for promoting adherence to handwashing guidelines is provided. The system includes: a sensor for detecting a handwashing event; a controller that receives signals from the sensor and activates a timer; a timer for sending an observable signal to a handwasher; and, a power source that provides power to components of the system.

In a method aspect of the present invention, a method for promoting adherence to handwashing guidelines is provided. The method includes the steps of: detecting a handwashing event using a sensor; transmitting a signal from the sensor to a controller that a handwashing event has occurred; and, transmitting a signal from the controller to a timer that initiates a countdown observable to a handwasher.

In another system aspect of the present invention, a system is provided for determining compliance with handwashing guidelines. The system includes: a sensor for detecting a handwashing event; a controller that receives signals from the sensor and activates a timer; a timer for sending an observable signal to a handwasher; a central processing unit for storing and analyzing data related to handwashing compliance; and, a power source that provides power to components of the system.

In another method aspect of the present invention, a method for determining compliance with handwasbing guidelines is provided. The methods includes the steps of: detecting a handwashing event using a sensor; transmitting a signal from the sensor to a controller that a handwashing event has occurred; transmitting a signal from the controller to a timer that initiates a countdown observable to a handwasher; determining whether a preset period for handwashing has been completed; and, transmitting data to a central processing unit related to whether the preset handwashing period has been completed.

### BRIEF DESCRIPTION OF THE DRAWINGS

FIG. 1 shows a system that provides feedback to a person who is washing his hands.

FIG. 2 shows a system that accumulates data and analyzes it to determine compliance with handwashing guidelines.

FIG. 3 shows a top view of a faucet spout (301) having a timer (302).

FIG. 4 shows a method of providing feedback to a person who is washing his hands.

FIG. 5 shows a method for the accumulation and analysis of data to determine compliance with handwashing guidelines.

### DETAILED DESCRIPTION OF THE INVENTION

The present invention generally relates to systems and methods to promote proper handwashing. Specifically, it relates to systems and methods that provide feedback to the person who is washing his hands. It further relates to systems and methods for monitoring hand hygiene compliance.

One might assume that a simple automated faucet—e.g., one with a preset period for handwashing—would significantly increase band hygiene. Such an assumption is, however, misplaced. A study was performed by researchers at Johns Hopkins University and Hospital on the effect of an automated sink on handwashing practices. See, Larson, E., et al. “Effect of an automated sink on handwashing practices and attitudes in high-risk units” Infect Control Hops. Epidemiol. 1991 Jul.;12(7):422-8. One thousand, six hundred and ten handwashes were observed over a two week period. Hospital staff expressed negative attitudes about the automated sink, resulting in significantly less handwashing over the course of the study.

The present invention applies a different approach, involving a handwashing system that does more than operate for a set period of time: It is a system that provides feedback to the handwasher, which makes it interactive. This interactive feature increases compliance.

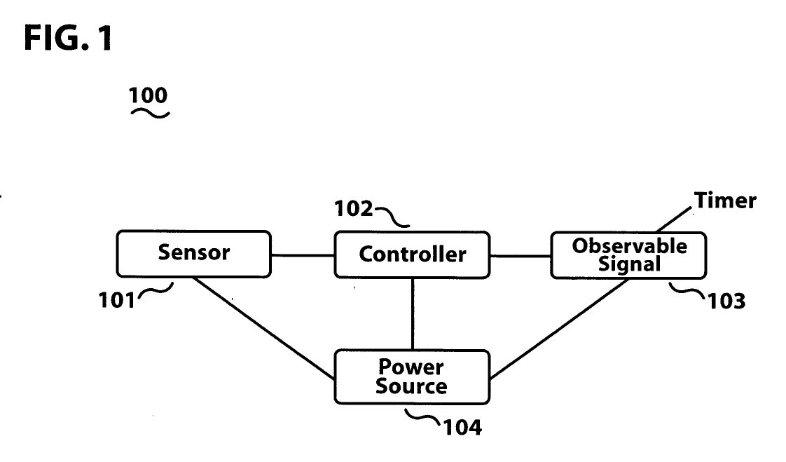

The present invention will be described in detail in reference to the figures. FIG. 1 shows a schematic representation of the subject handwashing system in its most general form. System 100 includes a sensor (101) that detects initiation of a handwashing event. Sensor 101 sends a signal to controller 102, which operates system 100 and timer 103. Timer 103 emits an observable signal to the handwasher that correlates with a preset period of time associated with adequate handwashing. System 100 is powered by power source 104.

Sensor 101 may be of any suitable design or mechanism. For instance, it may be an infiared detector, as discussed in U.S. Pat. Nos. 6,898,552, 6,294,786 and 6,161,814. It may alternatively be an ultrasonic detector, such as the one reported in U.S. Pat. No. 4,402,095. The detection may further be the result of mechanical actuation, such as that described in U.S. Pat. No. 6,883,541.

The initiating event, or “start handwashing event,” that is detected by the sensor may be of a variety of types including, but not limited to, the following: water starts to flow through a faucet; water hits the surface of a sink bowl; water passes through a sink drain; a person has triggered an infrared sensor to initiate water flow in the faucet by placing his hands under the faucet; and, a person has turned a faucet handle or faucet pedal to an on-position to initiate water flow through the faucet.

Controller 102 is typically a microcontroller or microprocessor. See U.S. Pat. No. 6,898,552 for a discussion of the use of microprocessors to control the operation of electronic circuitry involved in an automatic faucet. Controller 102 has several functions:. It detects whether there is enough power for system 100 to operate. If there is not, then it sends a signal turning off Sensor 101 and Timer 103. Controller 102 further receives and analyzes any signal it receives from Sensor 101. When it receives a “start handwashing event” signal, it initiates Timer 103; when it receives a “stop handwashing event” signal, it performs a check to determine whether Timer 103 is in an active mode.

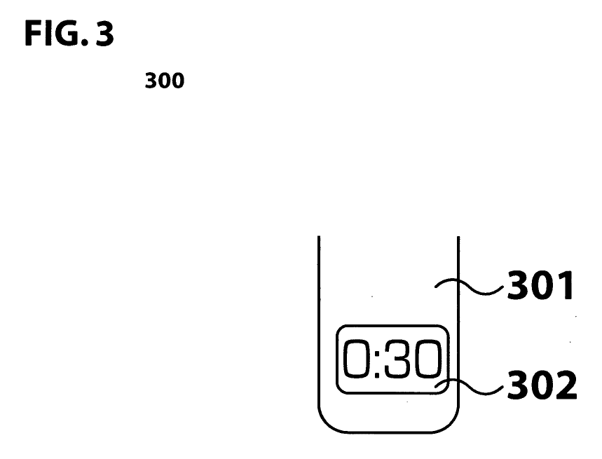

Timer 103 may be any device that can provide an observable signal to a handwasher that indicates whether a preset period of time (i.e., a period associated with proper handwashing and adequate hygiene) for handwashing has passed. In one embodiment, Timer 103 is a display placed in a position that is easily seen by the handwasher. Nonlimiting examples of Timer 103 placement include above or adjacent to the sink (e.g., a wall or mirror) in which handwashing occurs and on the faucet as shown in FIG. 3.

When Timer 103 is a display, it is typically designed to present bright, legible numbers corresponding to the preset period for handwashing. For example, if the preset period for handwashing is 30 seconds, the display would present the number “30” at the outset, and it would subsequently present a “countdown” upon initiation.

The observable signal produced by Timer 103 may further be of another visual type (e.g., a blinking light), may be auditory, or may be a mix of visual and auditory signals. Auditory signals include, without limitation, the following: a series of intermittent beeps lasting for the preset period; music that plays for the preset period; and, oral instructions that are provided during the preset period.

Power Source 104 may be any suitable power source, but is typically outlet power, batteries or solar cells. Power Source 104 should be long-lasting and should require minimal maintenance. If solar cells are used, they are typically regenerative.

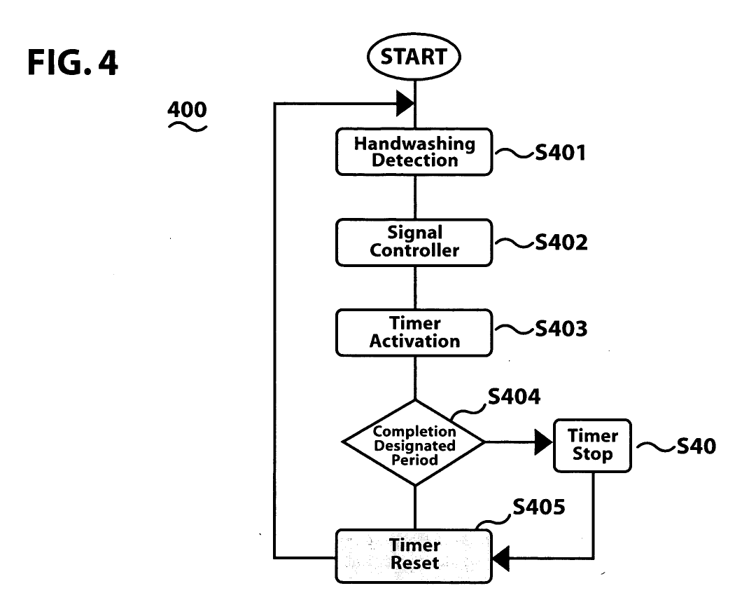

A method of using System 100 (400) is described in reference to FIG. 4, where Timer 103 is a display designed to present bright, legible numbers corresponding to the preset period for handwashing. A “start handwashing event” signal is detected by the sensor in S401. The signal is transmitted to the controller (S402), which activates the timer (S403) and initiates display countdown. If the preset period is 30 seconds, the display presents the number “30.” The countdown proceeds until either the 30 second period expires or the sensor detects a “stop handwashing event” signal. Nonlimiting examples of “stop handwashing event” signals include: water stops flowing through the faucet; water stops hitting the surface of the sink bowl; water stops passing through the sink drain; the handwasher has triggered an infrared sensor to stop water flow in the faucet by removing his hands from under the faucet; and, the handwasher has turned the faucet handle or faucet pedal to the off-position to stop water flow through the faucet.

The countdown is complete when the display reaches “0” (S404). At that time, the timer may optionally produce a completion signal (e.g., a loud, audible beep). The display continues to display “0,” until it receives a “stop handwashing event.” The timer is then reset (S405) to the preset time period.

Alternatively, if a “stop handwashing event” is detected before the countdown is complete, the controller halts the countdown (e.g., at “14”). After halting the countdown, the controller initiates an internal timer counting up from “0” until a second preset value is reached (e.g., “5”). Once the second preset value is reached, the internal timer stops counting and the controller resets the display timer (S405).

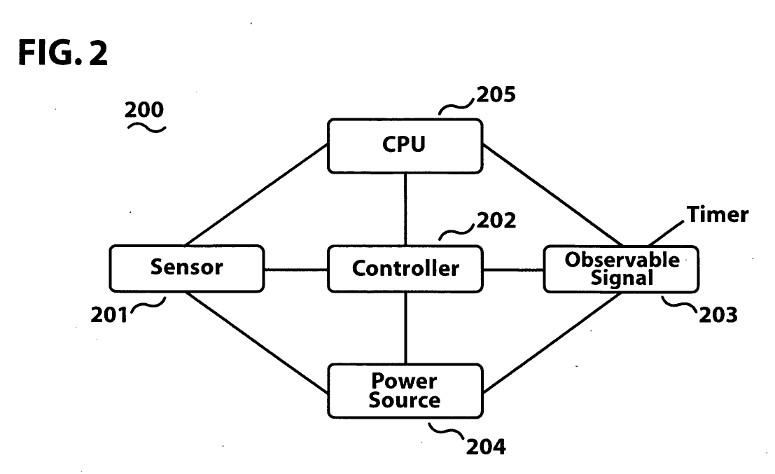

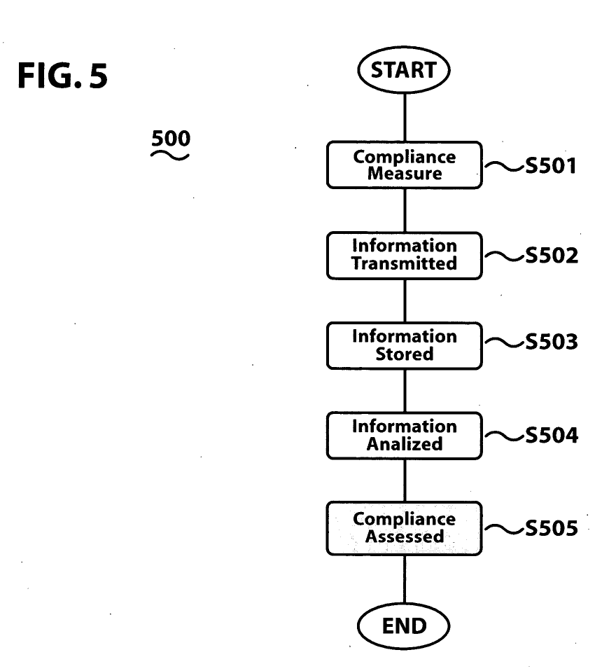

With respect to aspects of the present invention relating to systems and methods for monitoring hand hygiene compliance, the present invention will be described further in reference to FIGS. 2 and 5. System 200 of FIG. 2 is essentially the same as System 100 with the addition of a CPU. System 200 includes a sensor (201) that detects initiation of a handwashing event. Sensor 201 sends a signal to controller 202, which operates system 200 and timer 203. Timer 203 emits an observable signal to the handwasher that correlates with a preset period of time associated with adequate handwashing. System 200 is powered by power source 204. Data related to handwashing compliance is sent from controller 202 to CPU 205, either through wireless transmission or through any other suitable means.

FIG. 5 depicts the method of monitoring hand hygiene compliance using System 200. The controller determines whether a countdown is completed or interrupted (S501). This information is transmitted to the CPU in S502, where it is stored (S503). The information is analyzed (S504) to determine various correlations. Compliance is assessed from the various correlations (S505). Other results may be obtained from the analysis as well. Such results include, but are not limited to: if there is more than one handwashing site in a facility, determining whether compliance is better at one site than another, if there are sites associated with different professionals (e.g., doctors and nurses), determining whether compliance is better within one group than another.

The present systems and methods increase compliance with handwashing guidelines within a selected group. For instance, if a guideline states that handwashing with soap and water should occur for at least 30 seconds, the present systems and methods will increase compliance over a baseline measure at least 10 percent. Preferably, compliance is increased at least 20 or 30 percent. More preferably, compliance is increased 40 or 50 percent, and most preferably 60 or 70 percent.

The present systems and methods further provide facilities or organizations with a method for complying with the Center for Disease Control's recommendation that health care facilities develop and implement a system for measuring adherence to hand hygiene recommendations.

1. A system for promoting adherence to handwashing guidelines wherein the system comprises:
(a) a sensor for detecting a handwashing event;
(b) a controller that receives signals from the sensor and activates a timer;
(c) a timer for sending an observable signal to a handwasher; and,
(d) a power source that provides power to components of the system.

2. The system according to claim 1, wherein the sensor is and infrared sensor.

3. The system according to claim 1, wherein the controller is a microprocessor.

4. The system according to claim 1, wherein the timer is a display, and wherein the display presents numbers related to a preset period for handwashing.

5. The system according to claim 2, wherein the controller is a microprocessor, and wherein the timer is a display that presents numbers related to a preset period for handwashing.

6. A method for promoting adherence to handwashing guidelines, wherein the method comprises the steps of:
(a) detecting a handwashing event using a sensor;
(b) transmitting a signal from the sensor to a controller that a handwashing event has occurred; and,
(c) transmitting a signal from the controller to a timer that initiates a countdown observable to a handwasher.

7. The method according to claim 6, wherein the method further comprises a step of determining whether a preset period for handwashing has been completed.

8. The method according to claim 6, wherein if the preset period has been completed and handwashing has been stopped the timer is reset to the preset period.

9. The method according to claim 8, wherein the timer is on a surface adjacent to the place where handwashing is occurring.

10. The method according to claim 8, wherein the timer is on a faucet where handwashing is occurring.

11. A system for determining compliance with handwashing guidelines, wherein the system comprises:
(a) a sensor for detecting a handwashing event;
(b) a controller that receives signals from the sensor and activates a timer;
(c) a timer for sending an observable signal to a handwasher;
(d) a central processing unit for storing and analyzing data related to handwashing compliance; and,
(e) a power source that provides power to components of the system.

12. The system according to claim 11, wherein the sensor is and infrared sensor.

13. The system according to claim 11, wherein the controller is a microprocessor.

14. The system according to claim 11, wherein the timer is a display, and wherein the display presents numbers related to a preset period for handwashing.

15. The system according to claim 12, wherein the controller is a microprocessor, and wherein the timer is a display that presents numbers related to a preset period for handwashing.

16. A method for determining compliance with handwashing guidelines, wherein the method comprises the steps of:
(a) detecting a handwashing event using a sensor;
(b) transmitting a signal from the sensor to a controller that a handwashing event has occurred;
(c) transmitting a signal from the controller to a timer that initiates a countdown observable to a handwasher;
(d) determining whether a preset period for handwashing has been completed; and,
(e) transmitting data to a central processing unit related to whether the preset handwashing period has been completed.

17. The method according to claim 16, wherein the method further comprises the step of storing and analyzing data by the central processing unit.

18. The method according to claim 16, wherein the method further comprises the step of determining the percentage of handwashes that comply with the handwashing guidelines.

19. The method according to claim 16, wherein the method further comprises the step of determining whether a group of professionals is complying with the handwashing guidelines at a higher percentage than another group.

20. The method according to claim 17, wherein the method further comprises the step of determining the percentage of handwashes that comply with the handwashing guidelines.
